(19) United States
(12) Patent Application Publication
(10) Pub. No.: US 2005/0171390 A1
(43) Pub. Date: Aug. 4, 2005

(54) Wet oxidation process and system

(75) Inventors: Chad L. Felch, Mosinee, WI (US); Richard W. Lehmann, Birnamwood, WI (US); Todd J. Wingers, Rothschild, WI (US); Bruce L. Brandenburg, Wausau, WI (US); William M. Copa, Weston, WI (US)

Correspondence Address: LOWRIE, LANDO & ANASTASI, RIVERFRONT OFFICE, ONE MAIN STREET, ELEVENTH FLOOR, CAMBRIDGE, MA, 02142, US

(73) Assignee: USFilter Corporation, Warrendale, PA (US)

(21) Appl. No.: 11/014,619

(22) Filed: Dec. 16, 2004

Related U.S. Application Data
(60) Provisional application No. 60/530,369, filed on Dec. 17, 2003.

(51) Int. Cl. C07C 27/00

(52) U.S. Cl. 568/959

(57) ABSTRACT

A process and system for the destruction of compounds having a carbon-hetero atom bond. The process includes wet oxidation at elevated temperature and pressure of an aqueous mixture of at least one compound having a carbon-hetero atom bond to substantially destroy the carbon-hetero atom bond of the at least one compound. The resulting oxidized material may be further treated in an advanced oxidation process to destroy any residual carbon-hetero atom bonds remaining.

### RELATED APPLICATIONS

This application claims priority under 35 U.S.C. § 119(e) to U.S. Provisional Application Ser. No. 60/530,369 entitled “WET OXIDATION PROCESS AND SYSTEM,” filed on Dec. 17, 2003, which is herein incorporated by reference in its entirety for all purposes.

### BACKGROUND

1. Field of the Invention

The present invention relates to a wet oxidation system and process, and more particularly, to a subcritical wet oxidation process for destruction of specific chemical bonds.

2. Background

Wet oxidation is a well-known technology for the destruction of pollutants in wastewater. The process involves treatment of the wastewater with an oxidant, generally molecular oxygen from an oxygen-containing gas, at elevated temperatures and pressures.

Wet oxidation at temperatures below the critical temperature of water, 374° C., is termed subcritical wet oxidation. Subcritical wet oxidation systems operate at sufficient pressure to maintain a liquid water phase and may be used commercially for conditioning sewage sludge, the oxidation of caustic sulfide wastes, regeneration of powdered activated carbon, and the oxidation of chemical production wastewaters, to name only a few applications.

Complete mineralization of all pollutants in wastewater by wet oxidation may be achieved only with great difficulty. Toxic and hazardous compounds present in wastewater may be degraded to innocuous, biologically treatable substances by wet oxidation, however, in some instances, degradation products of wet oxidation may be resistant to biological treatment.

### BRIEF SUMMARY OF THE INVENTION

The invention is directed to a wet oxidation process for destruction of carbon-phosphorus, carbon-sulfur, and/or carbon-arsenic bonds comprising providing an aqueous mixture including at least one compound having a carbon-hetero atom bond, with the hetero atom is selected from the group consisting of phosphorus, sulfur and arsenic. The aqueous mixture pH is maintained between about 8 and about 14, and oxidized at an elevated temperature and superatmospheric pressure to substantially destroy the carbon-hetero atom bond of the at least one compound to form an alkaline oxidized mixture.

Another embodiment, is directed to a process for the destruction of carbon-hetero atom bonds comprising providing an aqueous mixture including at least one compound having a carbon-hetero atom bond, wherein the hetero atom is selected from the group consisting of: phosphorus, sulfur, and arsenic. The aqueous mixture is maintained at a pH between about 8 and about 14 and oxidized in a continuous process to destroy at least about 95% of the carbon-heteroatom bonds of the at least one compound. Oxidation occurs at a temperature of at least about 240° C. to less than about the critical temperature of water, at a pressure of at least about 33 atmospheres for about 1 hour to about 8 hours. In a further embodiment of the present invention, a two-stage oxidation process is employed for the destruction of carbon-phosphorus, carbon-sulfur, and/or carbon arsenic bonds. The process includes the steps of providing an aqueous mixture including at least one compound having a carbon-hetero atom bond, with the hetero atom selected from the group consisting of phosphorus, sulfur and arsenic. The aqueous mixture pH is maintained between about 8 and about 14, and the alkaline aqueous mixture is oxidized with a first oxidant at an elevated temperature and superatmospheric pressure to substantially destroy the carbon-hetero atom bond of the at least one compound to form a first alkaline oxidized mixture. The first oxidized mixture pH is adjusted to a range between about 3 and about 6 to produce an acidic first oxidized mixture. The acidic first oxidized mixture pH is then oxidized with a second oxidant to destroy the carbon-hetero atom bond of any of the at least one compound remaining therein.

Another embodiment of the invention is directed to a system for treating a source of an aqueous mixture comprising at least one compound having a carbon-hetero atom bond selected from the group consisting of phosphorus, sulfur and arsenic. A wet oxidation system is fluidly connected to the source, and an alkali source is disposed to introduce alkali upstream of the wet oxidation system.

Another embodiment of the invention is directed to a wet oxidation system comprising a source of an aqueous mixture, a wet oxidation system fluidly connected to the source of the aqueous mixture, including a reactor vessel, an alkali source fluidly connected to the source of the aqueous mixture and upstream of the wet oxidation system, and a source of carbonate fluidly connected to the rector vessel down stream of an inlet of the aqueous mixture to the reactor vessel.

Other advantages, novel features, and objects of the invention will become apparent from the following detailed description of non-limiting embodiments of the invention when considered in conjunction with the accompanying drawings, which are schematic and which are not intended to be drawn to scale. In the figures, each identical or nearly identical component that is illustrated in various figures typically is represented by a single numeral. For purposes of clarity, not every component is labeled in every figure, nor is every component of each embodiment of the invention shown where illustration is not necessary to allow those of ordinary skill in the art to understand the invention. In cases where the present specification and a document incorporated by reference include conflicting disclosure, the present specification shall control.

### BRIEF DESCRIPTION OF THE DRAWINGS

Preferred non-limiting embodiments of the present invention will be described by way of example with reference to the accompanying drawings, in which:

FIG. 1 is a system diagram of one embodiment of the wet oxidation system of the present invention.

FIG. 2 is a system diagram of another embodiment of the wet oxidation system of the present invention.

FIG. 3 is a system diagram of a further embodiment of the wet oxidation/advanced oxidation system of the present invention.

### DETAILED DESCRIPTION

The present invention relates to the treatment of wastewater containing carbon-phosphorus, carbon-sulfur, and or carbon-arsenic bonds. These bonds may be found in hazardous compounds such as chemical agents, pesticides, and herbicides which produce toxic effects on animal and plant biological systems. Many pesticides and herbicides contain chemical structures similar to those of the chemical agents.

Wastewaters containing chemical agents, or their degradation products, are generated when destroying outdated chemical weapons or in the planned reduction of existing military chemical weapons, and other wastewaters having such residuals. Chemical agents are generally highly reactive compounds, particularly with the biological systems of man and animals, producing toxic effects on such systems. Chemical agents include compounds termed nerve agents, including those having the phosphonofluoridate structure of the “G” series designation (GA, GB, GD, GF . . . ). Nerve agents may also include the phosphonothioate structure of the “V” series designation (VE, VG, VX, . . . ). Other chemical agents include the blister or vesicant agents containing carbon-sulfur bonds, such as sulfur mustards (H/HD, HT) and mustard-Lewisite (HL). Another class of chemical agents includes the blister or vesicant agents containing carbon-arsenic bonds, such as Adamsite (DM) (diphenylaminechloroarsine) and Lewisite (L) (2-chlorovinyl-dichloroarsine). Various pesticides and herbicides containing chemical structures similar to those of the chemical agents, as well as other chemical compounds containing carbon-phosphorus, carbon-sulfur, and/or carbon-arsenic bonds may be encountered in wastewaters or waste streams. The terms “wastewater” and “waste stream” are used interchangeably herein, referring to any type of source of water containing oxidizable constituents, including chemical compounds containing carbon-phosphorus, carbon-sulfur, carbon arsenic bonds, and combinations thereof. The source of water containing carbon-phosphorus, carbon-sulfur, carbon and/or carbon-arsenic bonds may take the form of direct piping from a plant or holding vessel.

To comply with various treaties on chemical agent destruction, it is desirable to disrupt one or more specific chemical bond in the chemical agent or degradation product(s) of the chemical agent. Conventional destruction of such chemical agents or their degradation products involves reacting the agents with water, alkali or an amino-alcohol, such as monoethanol amine (MEA). However, the resulting mixture typically includes carbon-phosphorus, carbon-sulfur, and/or carbon-arsenic bonds.

A method to destroy these bonds is dilution of the alkali or MEA mixture with water and oxidation of the resulting aqueous mixture. The oxidation of the aqueous alkali or aqueous MEA mixtures preferably converts any organo-phosphorus, organo-sulfur, and/or organo-arsenic compounds to ortho phosphate (PO₄), sulfate (SO₄), or arsenate (AsO₄), thereby destroying the carbon-phosphorus, carbon-sulfur, and/or carbon-arsenic bonds.

One aspect of the present invention involves a method for oxidative treatment of waste streams having carbon-phosphorus, carbon-sulfur, and or carbon-arsenic bonds, such as those found in chemical agent wastewaters, pesticide or herbicide wastewaters, to achieve a high Destruction Removal Efficiency (DRE) for the carbon-phosphorus, carbon-sulfur, and/or carbon-arsenic bonds of the compounds contained therein. As used herein, the phrase “high Destruction Removal Efficiency” is defined as destruction of at least about 95% of these bonds.

In one embodiment, an aqueous mixture of material including at least one compound having a carbon-phosphorus bond, a carbon-sulfur bond, and/or a carbon-arsenic bond is wet oxidized. The pH of the aqueous mixture is maintained or adjusted to a range of about 8 to about 14. In one embodiment, the pH of the aqueous mixture is maintained or adjusted to a range of about 9 to about 10.5, In another embodiment, the pH of the aqueous mixture is maintained or adjusted to a range of 10 or higher. The alkaline pH aqueous mixture is oxidized with an oxidant at an elevated temperature and superatmospheric pressure for a duration sufficient to substantially destroy the carbon-hetero atom bond of the at least one compound. As used herein, the phrase “substantially destroy” is defined as at least about 95% destruction. A hetero-atom is defined as any atom other than carbon or hydrogen. The process of the present invention is applicable to chemical agents, pesticides, herbicides, their degradation products, as well as other chemical compounds containing carbon-phosphorus, carbon-sulfur, and/or carbon-arsenic bonds.

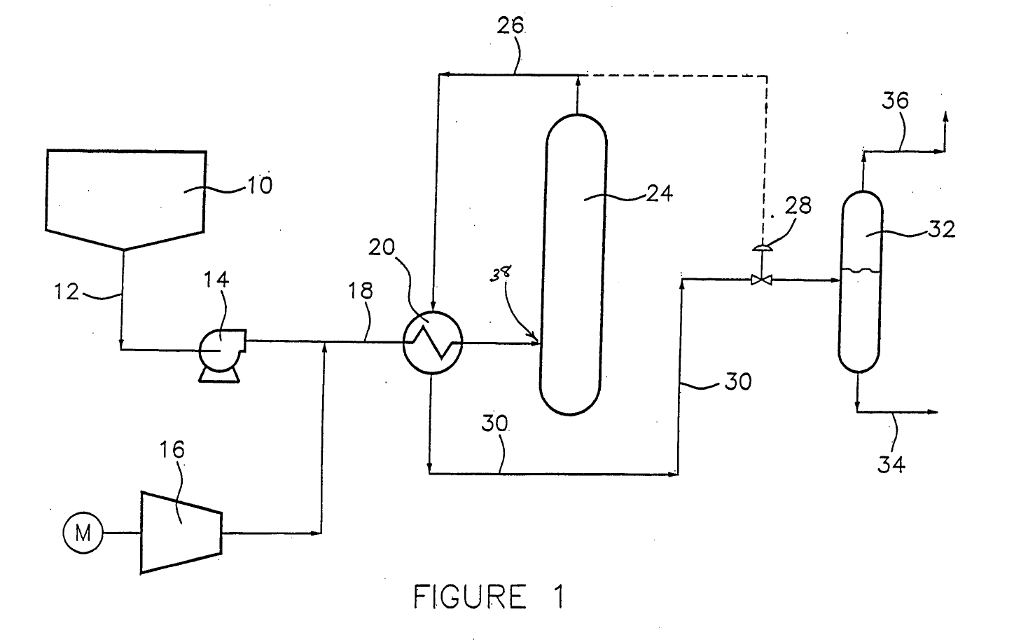

Wet oxidation may be performed in any known batch or continuous wet oxidation unit suitable for the compounds to be oxidized. Preferably, aqueous phase oxidation is performed in a continuous flow wet oxidation system, as shown in FIG. 1. Any oxidant may be used. Preferably, the oxidant is an oxygen-containing gas, such as air, oxygen-enriched air or essentially pure oxygen. As used herein, the phrase “oxygen-enriched air” is defined as air having an oxygen content greater than about 21%. Referring to FIG. 1, an aqueous mixture from a source, shown as storage tank 10 flows through a conduit 12 to a high pressure pump 14 which pressurizes the aqueous mixture. The aqueous mixture is mixed with a pressurized oxygen-containing gas, supplied by a compressor 16, within a conduit 18. The aqueous mixture flows through a heat exchanger 20 where it is heated to a temperature which initiates oxidation. The heated feed mixture then enters a reactor vessel 24 at inlet 38. Reactor vessel 24 provides a residence time wherein the bulk of the oxidation reaction occurs. The oxidized aqueous mixture and oxygen depleted gas mixture then exit the reactor through a conduit 26 controlled by a pressure control valve 28. The hot oxidized effluent traverses the heat exchanger 20 where it is cooled against incoming raw aqueous mixture and gas mixture. The cooled effluent mixture flows through a conduit 30 to a separator vessel 32 where liquid and gases are separated. The liquid effluent exits the separator vessel 32 through a lower conduit 34 while the gases are vented through an upper conduit 36.

In one embodiment, the wet oxidation process may be operated at a temperature below 374° C., the critical temperature of water. In a preferred embodiment, the wet oxidation process may be operated at a temperature between about 240° C. and about 373° C., and most preferably at a temperature between about 280° C. and about 350° C. In another embodiment, wet oxidation occurs at about 320° C. The retention time for the aqueous mixture at the selected oxidation temperature is preferably at least about 1 hour and up to about 8 hours. In one embodiment, the aqueous mixture is oxidized for about 1 hour to about 6 hours. In another embodiment, the aqueous mixture is oxidized for about 3 to about 6 hours. Sufficient oxygen-containing gas is supplied to the system to maintain an oxygen residual in the wet oxidation system offgas, and the gas pressure is sufficient to maintain water in the liquid phase at the selected oxidation temperature. For example, the minimum pressure at 240° C. is 33 atmospheres, the minimum pressure at 280° C. is 64 atmospheres, and the minimum pressure at 373° C. is 215 atmospheres. In one embodiment, the aqueous mixture is oxidized at a pressure of about 80 atmospheres to about 275 atmospheres.

Many of the chemical agents, or their precursor components in binary weapons, as well as pesticides and herbicides, contain halogen atoms such as fluorine or chlorine. Destruction of the compounds or their precursors by wet oxidation may generate halide anions. At acidic or near neutral pH, and at the elevated temperatures used for destruction of the carbon-phosphorus, carbon-sulfur, and/or carbon-arsenic bonds of the agents, the aqueous oxidation mixture is corrosive to the materials of construction of the wet oxidation system. Consequently, in one embodiment, the feed mixture may contain sufficient caustic material to maintain an alkaline pH, preferably above about pH 8, during the wet oxidation process. A material of construction suitable for the wet oxidation system operated at highly alkaline pH and temperatures in excess of 240° C. is a nickel-base alloy, such as alloy 600.

The present invention promotes treatment of the aqueous material. Any caustic substance may be used to maintain the aqueous material within a pH range of 8 to 14. In one embodiment, a metal hydroxide may be used, preferably, an alkali metal hydroxide, such as sodium hydroxide or potassium hydroxide. In practicing the process of the present invention, the carbonaceous portion of the agents or precursors is oxidized to carbon dioxide, much of which is retained in the alkaline solution as metal carbonate/bicarbonate salts. The alkaline earth metal hydroxides, such as calcium hydroxide, form insoluble carbonate salts. Thus, alkali metal hydroxides are most preferred for adjusting the feed mixture pH to the range of about 8 to about 14. In one embodiment, sodium hydroxide is used to increase the pH of the aqueous material. It is noted that an oxidation product of sodium hydroxide is sodium carbonate, which can become less soluble as temperature increases. As noted, potassium hydroxide may also be used to increase the pH of the aqueous material. An oxidation product of potassium hydroxide is potassium carbonate, which is more soluble than sodium carbonate at elevated temperatures.

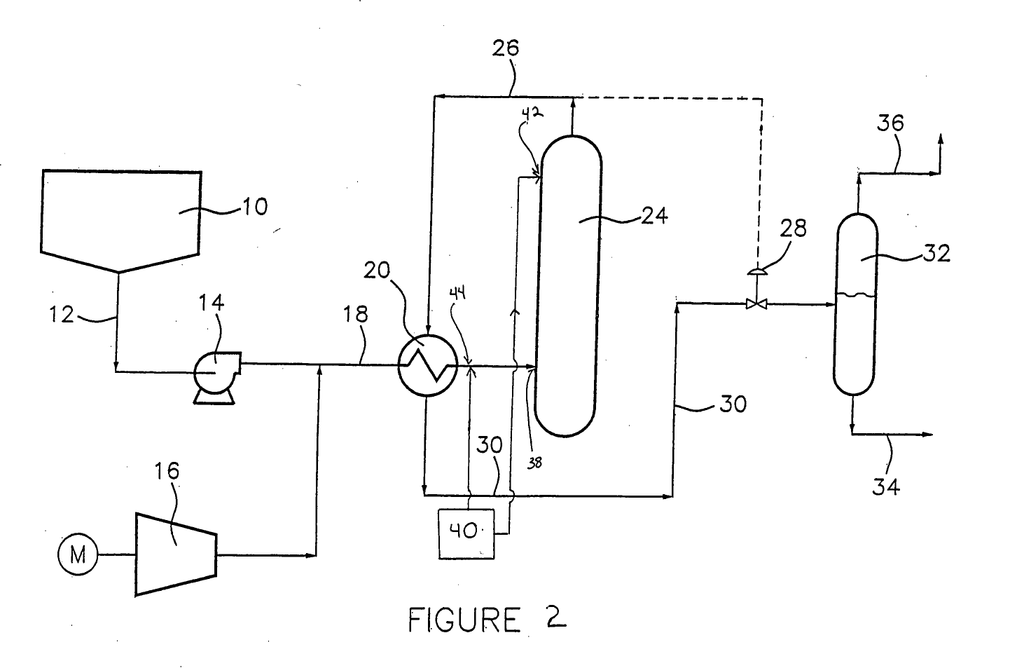

In another embodiment of the present invention, carbonate/bicarbonate may be added to the aqueous mixture prior to, or during oxidation. In one embodiment, carbonate/bicarbonate may be added to a wet oxidation process to increase oxidation efficiencies. In another embodiment, carbonates/bicarbonates may be added to the wet oxidation process to increase the destruction efficiency of carbon-phosphorus, carbon-sulfur, and/or carbon-arsenic bonds. As shown in FIG. 2, a source of carbonate 40 feeds into the aqueous mixture at inlet 44 and/or may feed directly into reactor vessel 24 at inlet 42. As used herein, “a source of carbonate” is defined as carbonate, bicarbonate, and/or any chemical compound that forms a carbonate upon oxidation. Inlet 42 may be positioned in reactor vessel 24 at any location to allow sufficient time for the oxidation reaction to be completed or nearly completed. Any carbonate, such as sodium carbonate and/or potassium carbonate may be used. In another embodiment, a source of carbonate includes, but is not limited to, inorganic material which in the oxidation process forms a carbonate/bicarbonate ion. For example, a metal hydroxide, such as sodium hydroxide and or potassium hydroxide may be added to the wet oxidation process. Addition of a source of carbonate may occur at any point before and/or during oxidation of the aqueous mixture, although it is preferred that the carbonate/bicarbonate ions be present in the aqueous mixture early enough to allow time for the oxidation reactions to be completed or nearly completed. In a preferred embodiment, carbonate is added directly into reactor vessel 24 at inlet 42, thus allowing sufficient time for oxidation to occur at a later stage in the oxidation process.

In another embodiment of the present invention, extraneous organic matter that produces carbonate/bicarbonate ions upon combustion may be added to the aqueous mixture prior to or during oxidation. Any organic matter that may readily be oxidized to carbonate may be used. In one embodiment, compounds having the phenolic —OH functional group or the quinine structure are easily oxidized to carbonate in an alkaline environment. Examples of organic matter include, but are not limited to, phenol, ethanol, cresol, quinine, hydroquinone, anthraquinone, and combinations thereof. In one embodiment, phenol is added directly into reactor vessel 24 at inlet 42 to allow sufficient time for oxidation to the carbonate as well as sufficient time for oxidation of any remaining aqueous material not previously oxidized in an earlier stage of the oxidation process.

In addition to facilitating operation of the wet oxidation system at the elevated temperatures and pressures, the addition of organic matter, surprisingly increases the destruction of the carbon-phosphorus, carbon-sulfur, and/or carbon-arsenic bonds of the aqueous material. The advantage of the addition of extraneous organic matter to dilute wastewaters is illustrated in Example IV and Table IV, as well as Example VIII and Table VIII.

In yet another embodiment of the present invention, the effluent from the wet oxidation process may be further treated in an advanced oxidation (AO) step to destroy the carbon-phosphorus, carbon-sulfur, and/or carbon-arsenic bonds of any remaining substance therein. The advanced oxidation step consists of further oxidation of the effluent with ozone (O₃), hydrogen peroxide (H₂O₂), ultraviolet light (UV), or combinations thereof. In one embodiment of the invention, the advance oxidation step may be a Fenton's Reagent Oxidation Process including acidifying the alkaline wet oxidation effluent to about pH 3 to about 6, then oxidation treatment with Fenton's Reagent. As used herein, Fenton's Reagent is defined as an iron-catalyzed hydrogen peroxide. The iron catalyst may be a Fe²⁺ or Fe³⁺ salt. In a preferred embodiment, ferrous salt is used as a catalyst for hydrogen peroxide. Most preferably, the advanced oxidation step includes oxidation treatment of the wet oxidation effluent with ozone and UV light. Such advanced oxidation treatment is carried out in a vessel or tank at or near ambient temperature and pressure. In a preferred embodiment of the AO treatment system, the alkaline wet oxidation effluent is first adjusted to a pH of 4 to 5 with mineral acid, then sparged with air to remove carbonate (as CO₂), prior to treatment with ozone and UV light. The advance oxidation treated effluent optionally is adjusted to near neutral pH prior to discharge.

Bench Scale Wet Oxidation (Autoclave) Reactors

Bench scale wet oxidation tests were performed in laboratory autoclaves. The autoclaves differ from the full scale system in that they are batch reactors, where the full scale unit may be a continuous flow reactor. The autoclaves typically operate at a higher pressure than the full scale unit, as a high charge of air must be added to the autoclave in order to provide sufficient oxygen for the duration of the reaction. The results of the autoclave tests provide an indication of the performance of the wet oxidation technology and are useful for screening operating conditions for the wet oxidation process.

The autoclaves used were fabricated from alloy 600 and Nickel 200. The selection of the autoclave material of construction was based on the composition of the wastewater feed material. The autoclaves selected for use, each have total capacities of 500 or 750 ml.

The autoclaves were charged with wastewater and sufficient compressed air to provide excess residual oxygen following the oxidation (ca. 5%). The charged autoclaves were placed in a heater/shaker mechanism, heated to the desired temperature (280° C.-350° C.) and held at temperature for the desired time, ranging from about 60 minutes to about 360 minutes.

During the heating and reacting periods, the autoclave temperature and pressure were monitored by a computer controlled data acquisition system. Immediately following oxidation, the autoclaves were removed from the heater/shaker mechanism and cooled to room temperature using tap water. After cooling, the pressure and volume of the off gas in the autoclave head-space were measured. A sample of the off-gas was analyzed for permanent gases. Subsequent to the analysis of the off gas, the autoclave was depressurized and opened. The oxidized effluent was removed from the autoclave and placed into a storage container. A portion of the effluent was submitted for analysis and the remaining sample was used for post-oxidative treatment. In order to generate sufficient volume for analytical work and post-oxidation test work, multiple autoclave tests for each condition were run.

The function and advantages of these and other embodiments of the present invention will be more fully understood from the following examples. These examples are intended to be illustrative in nature and are not considered to be limiting the scope of the invention. In the following examples, simulant compounds having carbon-phosphorus and/or carbon-sulfur bonds similar to those of chemical agents, pesticides, herbicides or their precursors are treated by wet oxidation to affect destruction of carbon-phosphorus and/or carbon-sulfur bonds therein. The resulting wet oxidation alkaline effluent optionally may be further treated by advanced oxidation to destroy the carbon-phosphorus and/or carbon-sulfur bonds of any remaining substances therein, if present.

### EXAMPLE I

Dimethyl methylphosphonate, H₃C—PO(OCH₃)₂, was used as a simulant for compounds containing a carbon-phosphorus bond such as H₃C—POF₂. A solution of dimethyl methylphosphonate (DMMP) in distilled water was prepared. The solution contained 20,000 mg/l DMMP and had a pH of 4.16. In tests No. 3 and 4, the solution pH was adjusted to 14 with sodium hydroxide. A sample of the solution was placed in an autoclave and pressurized with air to provide sufficient oxygen to oxidize all the oxygen demand of the solution, as well as sufficient overpressure to maintain water in the liquid phase during heating. The autoclave was heated to temperature for one hour, then cooled, and the gas and liquid phases analyzed with the results shown in Table I.

TABLE I
DMMP Oxidation At Temperature For 1 Hour

| | | Test No. | | | |
|---|---|---|---|---|---|
| | FEED | 1 | 2 | 3 | 4 |
| Temp., ° C. | — | 260 | 280 | 260 | 280 |
| pH | 4.16/14.0 | 1.96 | 1.97 | 13.43 | 11.87 |
| DMMP, | 20,000 | — | — | — | — |
| mg/L | | | | | |
| MPA, mg/L | <8 | 13,100 | 12,400 | 12,700 | 11,300 |
| Ortho-P, | <0.5 | 66 | 134 | 394 | 654 |
| mg/L | | | | | |
| NPOC, mg/L | 5310 | 4602 | 4555 | 4505 | 3870 |

After wet oxidation at these temperatures and pH levels, DMMP was below detectable levels. Although DMMP was extensively destroyed at all temperatures and pH tested, the majority of the carbon-phosphorus bonds remained in tact as methylphosphonic acid (MPA). Higher temperatures and alkaline pH provided improved, but relatively low, formation of ortho phosphate. MPA destruction was relatively low, although improved at higher temperatures and alkaline pH, as well.

### EXAMPLE II

To further investigate the destruction of carbon-phosphorus bonds, methylphosphonic acid, H₃C—PO(OH)₂, was used as a simulant compound. A solution of methylphosphonic acid (MPA) in distilled water was prepared. The solution contained 5,000 mg/l MPA and had a pH of 1.7 A sample of the solution was placed in an autoclave and pressurized with air to provide sufficient oxygen to oxidize all the oxygen demand of the solution, as well as sufficient overpressure to maintain water in the liquid phase during heating. The autoclave was heated to 320° C. for selected time periods, then it was cooled and the liquid phase analyzed with the results shown in Table II.

TABLE II
MPA Oxidation At 320° C.

| | | Test No. | | |
|---|---|---|---|---|
| | FEED | 5 | 6 | 7 |
| Time, minutes | — | 60 | 180 | 360 |
| pH | 1.7 | 1.9 | 2.16 | 1.45 |
| MPA, mg/L | 5,000 | — | 2,300 | — |
| Organic-P, mg/L | 1,800 | 1,240 | — | <10 |
| Ortho-P, mg/L | <3.5 | 490 | 689 | 1,330 |
| Total P, mg/L | 1,800 | 1,740 | — | 1,340 |

Methyl phosphonic acid was only partially destroyed after one hour at temperature, as indicated by the small fraction of ortho phosphate formed and the large fraction of organic phosphorus remaining. After 3 hours at temperature, only about 54% of the MPA was destroyed and about 52% of the total phosphorus was converted to ortho phosphorus. Complete destruction of MPA was achieved after six hours at temperature where no detectable organic phosphorus remained and ortho phosphorus equaled total phosphorus.

### EXAMPLE III

To further investigate the effect of high pH on the destruction of carbon-phosphorus bonds of Examples I and II, a solution of methylphosphonic acid (MPA) in distilled water was prepared. The solution contained 5,000 mg/l MPA and the pH of the solution adjusted to 12.8 with sodium hydroxide. A sample of the solution was placed in an autoclave and pressurized with air to provide sufficient oxygen to oxidize all the oxygen demand of the solution, as well as sufficient overpressure to maintain water in the liquid phase during heating. The autoclave was heated to temperature for selected time periods, then cooled and the liquid phase analyzed with the results shown in Table III.

TABLE III
MPA Oxidation at pH 12.8

| | | Test No. | | | |
|---|---|---|---|---|---|
| | FEED | 8 | 9 | 10 | 11 |
| Time, minutes | — | 60 | 60 | 60 | 360 |
| Temp., ° C. | — | 280 | 300 | 320 | 320 |
| pH | 12.8 | 12.4 | 12.3 | 11.4 | 9.2 |
| MPA, mg/L | 5,000 | 4,000 | 3,120 | 1,780 | 125 |
| Ortho-P, | 2.0 | 172 | 471 | 932 | 1,350 |
| mg/L | | | | | |

MPA was analyzed using ion chromatography. The MPA analyses and the distribution of the phosphorus at the various temperatures and duration tested show that high temperature and extended oxidation times achieve high degrees of destruction for carbon-phosphorus bonds for liquids at elevated pH. Increasing the temperature from 280° C. to 320° C. increased the destruction of MPA at one hour from about 20 percent to about 64 percent. Increasing the wet oxidation time from 1 hour to about 6 hours at 320° C., increased the destruction of MPA from about 64 percent to about 97.5 percent.

### EXAMPLE IV

Dimethyl methylphosphonate, H₃C—PO(OCH₃)₂, was used as a stimulant compound containing a carbon-phosphorus bond and dimethyl sulfoxide, H₃C—SO—CH₃ (DMSO), was used as a simulant compound containing a carbon-sulfur bond. A solution of dimethyl methylphosphonate (DMMP) and dimethyl sulfoxide (DMSO) in distilled water was prepared. The solution contained 1,249 mg phosphorus/L from DMMP and 2,053 mg sulfur/L from DMSO. Phenol at 4,631 mg/L was added as extraneous organic matter. Tests were performed on portions of the feed at an alkaline, a neutral and an acidic pH. A sample of the solution was placed in an autoclave and pressurized with air to provide sufficient oxygen to oxidize all the oxygen demand of the solution, as well as sufficient overpressure to maintain water in the liquid phase during heating. The autoclave was heated to 280° C. for one hour, then cooled. The gas and liquid phases were analyzed with the results shown in Table IV.

TABLE IV
DMMP/DMSO/PHENOL Oxidation at 280° C. for 1 Hour

| | | Test No. | | |
|---|---|---|---|---|
| | FEED | 12 | 13 | 14 |
| pH | | 13.5 | 6.5 | 4.4 |
| DMMP-Phos., | 1,249 | — | — | — |
| mg/L | | | | |
| MPA-Phos., mg/L | 0 | 949 | 810 | 874 |
| Ortho-Phos, mg/L | 0 | 216 | 219 | 193 |
| C—P Bond, % DRE | — | 24 | 35.2 | 30 |
| DMSO-Sulfur, | 2,053 | — | — | — |
| mg/L | | | | |
| MSA-Sulfur, mg/L | 0 | 945 | 385 | 310 |
| SO₄-Sulfur, mg/L | 0 | 842 | 1553 | 1615 |
| C—S Bond, % DRE | — | 53.9 | 86.1 | 85.4 |
| Phenol, mg/L | 4,631 | 45 | 63 | 24 |

DMMP was extensively destroyed at all temperatures tested, however only moderate destruction of the carbon-phosphorus bond to produce ortho phosphate was obtained. The methylphosphonic acid (MPA) formed is moderately stable to wet oxidation at the conditions tested, even with phenol added. In contrast, significant destruction of the carbon-sulfur bonds of DMSO and methylsulfonic acid (MSA) was achieved at the test conditions with phenol added.

### EXAMPLE V

In order to simulate the composition of chemical agent H/HD (Bis-(2-chloroethyl) sulfide) in MEA, a stock stimulant solution was prepared. The stock stimulant solution contained, on a weight percent basis, 83.0% ethanolamine, 10.1% distilled water, 3.90% 1,2 dichloroethane, and 3.00% dimethyl sulfoxide. The stock stimulant solution was diluted 50:1 with distilled water for use in autoclaving testing. Solid sodium hydroxide was added to the diluted solution to yield 48 g/L NaOH in the solution. A sample of the diluted solution was placed in an autoclave and pressurized with air to provide sufficient oxygen to oxidize all the oxygen demand of the solution, as well as sufficient overpressure to maintain water in the liquid phase during heating. The autoclave was heated to temperature for one hour, then cooled and the liquid phase analyzed with the results shown in Table V.

TABLE V
H Simulant in MEA Oxidation at Temperature for 1 Hour

| | | Test No. | | |
|---|---|---|---|---|
| | FEED | 15 | 16 | 17 |
| Temp., ° C. | — | 280 | 300 | 320 |
| pH | >10 | 9.8 | 9.8 | 10.4 |
| C—S DRE, % | — | 30.9 | 51.8 | 71.7 |
| Total-S. mg/L | 247 | — | — | — |
| SO₄—S, mg/L | — | 76 | 128 | 177 |

The calculated C—S bond DRE for the wet oxidation step ranged from about 31% at the lowest temperature of 280° C. to 71.7% at the highest temperature of about 320° C. The concentration of sulfate in the oxidized effluents increased with increasing reaction temperature from 76 mg/L at the lowest temperature to 177 mg/L at the highest. These results indicate an increase in C—S bond oxidation to form sulfate with increasing oxidation temperature.

### EXAMPLE VI

In order to simulate the composition of GB (Isopropyl methyl phosphonofluoridate, commonly known as Sarin) in MEA, a stock stimulant solution was prepared. The stock stimulant solution contained, on a weight percent basis, 39.5% ethanolamine, 52.0% distilled water, 6.9% dimethyl methylphosphonate, and 1.6% hexafluorobenzene. The stock stimulant solution was diluted 12:1 with distilled water for use in autoclave testing. Solid sodium hydroxide or concentrated sulfuric acid was added to the diluted solution to control the pH of the oxidized effluent. A sample of the diluted solution was placed in an autoclave and pressurized with air to provide sufficient oxygen to oxidize all oxygen demand of the solution, as well as sufficient overpressure to maintain water in the liquid phase during heating. The autoclave was heated to temperature for the selected time duration, and then cooled. The liquid phase was analyzed with the results shown in Table VI.

TABLE VI
GB Simulant in MEA Oxidation

| | | Test No. | | | | | | | | |
|---|---|---|---|---|---|---|---|---|---|---|
| | FEED | 18 | 19 | 20 | 21 | 22 | 23 | 24 | 25 | 26 |
| Temp., ° C. | — | 280 | 280 | 280 | 280 | 300 | 320 | 320 | 320 | 320 |
| Time, mins. | — | 60 | 180 | 60 | 180 | 120 | 60 | 180 | 60 | 180 |
| pH | 10.8 | 9.9 | 9.4 | 5.5 | 5.9 | 8.9 | 9.3 | 9.3 | 5.5 | 6.9 |
| MPA, mg/L | 4,450* | 2,616 | 558 | 2,508 | 1,445 | 1,650 | 49 | 36 | 1,120 | 825 |
| C-P DRE, % | — | 41.2 | 87.5 | 43.6 | 67.5 | 62.9 | 98.9 | 99.2 | 74.8 | 81.5 |

*Calculated MPA concentration from DMMP in the feed.

Analyses of the oxidized effluent in test Numbers 18-26 for methylphosphonic acid were performed using ion chromatography. MPA, the intermediate formed before final C—P bond destruction, was extensively destroyed at higher temperatures and at alkaline pH. The MPA concentrations were used to calculate C—P bond DRE values. At 320° C. and a pH of 9.3, the percent Destruction Removal Efficiency of the carbon-phosphorus bond reached 98.9% at one hour, and 99.2% at three hours.

### EXAMPLE VII

In order to simulate the composition of DF (methylphosphonyldifluoride), a stock simulant solution was prepared. The stock simulant solution contained, on a weight percent basis, 66.7% dimethyl methylphosphonate and 33.3% hexafluorobenzene. The simulant stock solution of DF was diluted 100:1 with distilled water within the autoclave for testing. Solid sodium hydroxide was added to the diluted solution to yield 34.7 g/L NaOH in the solution. A sample of the diluted solution in an autoclave was pressurized with air to provide sufficient oxygen to oxidize all the oxygen demand of the solution, as well as sufficient over pressure to maintain water in the liquid phase during heating. The autoclave was heated to temperature for the selected time duration, then cooled and the liquid phase analyzed with the results shown in Table VII.

TABLE VII
DF Simulant Oxidation At pH > 10.0

| | | Test No. | | |
|---|---|---|---|---|
| | FEED | 27 | 28 | 29 |
| Temp., ° C. | — | 320 | 320 | 350 |
| Time, minutes | — | 180 | 360 | 180 |
| pH | >10.0 | 10.5 | 10.1 | 10.2 |
| MPA, mg/L | 6,500* | 70.0 | 7.0 | 64.0 |
| C—P DRE, % | — | 98.94 | 99.89 | 99.02 |

*Calculated MPA concentration from DMMP in the feed

Analyses of the oxidized effluent in test Numbers 27-29 for methylphosphonic acid, performed using ion chromatography, were used to calculate C—P bond DRE values. At 320° C., the DRE averaged 98.94 percent at 3 hours, and increased to 99.89 percent at 6 hours. At 350° C., the DRE was 99.02% at 3 hours. Thus, a high degree of destruction of the C—P bond in the simulant DMMP has been demonstrated.

### EXAMPLE VIII

The DF simulant stock solution of Example VII was diluted 100:1 with distilled water within the autoclave for testing. Solid KOH was added to the diluted solution to yield about 40 g/L KOH. Phenol at 4600 mg/L was added as extraneous organic matter in tests No. 31 and 33. A sample of the diluted solution in an autoclave was pressurized with air to provide sufficient oxygen to oxidize all the oxygen demand of the solution, as well as sufficient over pressure to maintain water in the liquid phase during heating. The autoclave was heated to temperature for the selected time duration, then it was cooled and the liquid phase analyzed with the results shown in Table VII.

TABLE VIII
DF Simulant Oxidation With Phenol At 320° C.

Test No

| | FEED | 30 | 31 | 32 | 33 |
|---|---|---|---|---|---|
| Time, min. | — | 120 | 120 | 180 | 180 |
| pH | >10.0 | 9.7 | 9.3 | 9.2 | 9.3 |
| Phenol | — | No | Yes | No | Yes |
| Added | | | | | |
| MPA, mg/L | 7350* | 5732 | 147 | 1810 | 81 |
| MPA-P, | 2373* | 1850 | 47 | 584 | 26 |
| mg/L | | | | | |
| C-P DRE, % | — | 22.0 | 98.0 | 75.4 | 98.9 |

*Calculated MPA and MPA-P concentrations from DMMP in the feed

Again, analyses of the oxidized effluent in test Numbers 30-33 for methylphosphonic acid, performed using ion chromatography, were used to calculate C—P bond DRE values. Again, whether extraneous organic matter was added or not, the DRE showed an increase when oxidation time was increased from 2 to 3 hours. The presence of extraneous organic matter improved the percentage destruction of carbon-phosphorus bonds at wet oxidation durations of both 120 and 180 minutes, reaching 98% and 98.9%, respectively.

### EXAMPLE IX

In order to simulate the composition of QL (O-ethyl O-2-diisopropylaminoethyl methylphosphonite), a stock simulant solution was prepared. The stock simulant solution contained, on a weight percent basis, 39.79% dimethyl methylphosphonate and 60.21% dibutyl amine. The simulant stock solution of QL was diluted 125:1 with distilled water within the autoclave for testing. Solid sodium hydroxide was added to the diluted solution to yield 28.0 g/L NaOH in the solution. A sample of the diluted solution in an autoclave was pressurized with air to provide sufficient oxygen to oxidize all the oxygen demand of the solution, as well as sufficient overpressure to maintain water in the liquid phase during heating. The autoclave was heated to temperature for the selected time duration, then it was cooled and the liquid phase analyzed with the results shown in Table IX.

TABLE IX
QL Simulant Oxidation At pH > 10.0

| | | Test No. | | |
|---|---|---|---|---|
| | FEED | 34 | 35 | 36 |
| Temp., ° C. | — | 300 | 320 | 350 |
| Time, minutes | — | 360 | 180 | 360 |
| pH | >10.0 | 10.15 | 10.5 | 10.5 |
| MPA, mg/L | 2135* | 38.0 | <10.0 | <10.0 |
| C—P DRE, % | — | 98.22 | >99.5 | >99.5 |

*Calculated MPA concentration from DMMP in the feed.

Analyses of the oxidized effluent in test Numbers 34-36 for methylphosphonic acid, performed using ion chromatography, were used to calculate C—P bond DRE values. At 300° C. and 6 hours, the DRE of the C—P bond is greater than 98%. At 320° C. for 3 hours the DRE of the C—P bond was greater than 99.5%. Similarly, at 350° C. for 6 hours, the DRE of the C—P bond was greater than 99.5%. Thus, a high degree of destruction of the C—P bond in the simulant DMMP has been demonstrated.

Delayed Injection of Organic Material

Delayed injection of a readily oxidizable material may be used to enhance the destruction efficiency in any wet oxidation process in a later stage of the oxidation process. Although not bound by any particular theory, readily oxidizable materials may give rise to an increased concentration of oxidation radicals, such as hydroxyl OH. and hydroperoxyl HO₂. free radicals. These radicals may enhance the overall destruction efficiency of a targeted compound. If the aqueous mixture initially includes the targeted compound and also other oxidizable material, the easily oxidizable materials are the first to be oxidized and may produce an environment within the reactor, for example, at the bottom of a bubble column reactor, that is conducive to oxidation of all oxidizable materials present. As the oxidation reaction proceeds, the concentration of the easily oxidizable materials is depleted and free radical chain terminating reactions reduce the concentration of the oxidation radicals, hydroxyl OH. and hydroperoxyl HO₂. free radicals. This reduction may result in a decrease in the rate at which other compounds, which may be more difficult to oxidize, may be destroyed. These compounds may then be present in the oxidized effluent at higher concentrations than desired. The injection of an easily oxidizable material into the reactor at a point where the concentration of easily oxidizable components that were initially present in the aqueous mixture has been depleted, may also again increase the concentration of the oxidation radicals and a higher rate of reaction of the targeted compound may be maintained in the latter stages of the oxidation reaction.

### EXAMPLE X

Control and test wet oxidation runs were made using shaking autoclaves having a volume of 750 mL and a liquid charge volume of 150 mL. In both the control and test runs, QL hydrolysate which contains carbon-phosphorus bonded compounds as well as other extraneous, easily oxidizable materials were oxidized for a total of 180 minutes at 320° C. under similar conditions. However, in the test run, phenol was injected into the autoclave after 120 minutes, for further oxidation for an additional 60 minutes.

In the control run, a diluted sample of QL hydrolysate was charged to the autoclave, and the pH was maintained between 9 and 10. The control was run without the delayed addition of phenol. The autoclave was charged with compressed air, heated to 280° C., and cooled to room temperature using a cold water quench. Offgas # 1 from the autoclave was discharged after measuring the residual oxygen concentration. The autoclave was re-charged with a fresh sample of compressed air, heated to 320° C. for 180 minutes, and cooled to room temperature using a cold water quench. Offgas #2 was discharged after measuring the residual oxygen concentration. The oxidized effluent was removed from the autoclave and submitted for chemical analyses. The destruction efficiency of the C—P bond was 98.65% and the total organic carbon (TOC) was 57 mg/L.

A test with the delayed addition of phenol was run. An amount of diluted QL hydrolysate and air equal to that of the control was charged to the autoclave, and the pH maintained between 9 and 10. The autoclave was heated to 280° C. and cooled to room temperature using a cold water quench. Offgas #1 was discharged after measuring the residual oxygen concentration. The autoclave was recharges with a fresh sample of compressed air, heated to 320° C. for 120 minutes, and cooled to room temperature with a cold water quench. Offgas # 2 was discharged after measuring the residual oxygen concentration. An equivalent of 20 g/L of phenol was added to the partially oxidized QL hydrolysate in the autoclave. The autoclave was again charged with compressed air, heated to 280° C., and cooled to room temperature with a cold water quench. Off gas #3 was discharged after the residual oxygen concentration was measured. The autoclave was again recharged with compressed air, heated to 320° C. for 60 minutes, and cooled to room temperature with a cold water quench. Offgas #4 was discharge after measuring the residual oxygen concentration. The oxidized effluent was removed from the autoclave and submitted for chemical analyses. The destruction efficiency of the C—P bond was 99.85%, and the TOC was 41 mg/L.

TABLE X
QL Simulant Oxidation with Addition of Phenol

| Test No. | Feed | No Phenol | Phenol added |
|---|---|---|---|
| pH | — | 9.5 | 9.63 |
| Temp., ° C. | — | 320 | 320 |
| Total Time, minutes | — | 180 | 180 |
| TOC mg/L | 13,450 | 57 | 41 |
| Phosphorus MP and MPA, mg/L | 2,258 | 30 | 3 |
| C—P Bond Destruction, % | — | 98.65 | 99.85 |

The delayed injection of phenol improved the destruction efficiency of the C—P bonds, increasing the destruction efficiency from 98.65% to 99.85%. Moreover, even with the addition of phenol, the TOC decreased from 57 mg/L to 41 mg/L.

Post-Oxidation Carbonate Removal

Prior to UV/O₃ treatment, it is preferable to remove the carbonates from the wet oxidized effluent in order for the advanced oxidation (AO) processes to be effective. In one embodiment carbonate may be removed by adjusting the wet oxidation effluent to a pH of about 4 to about 5 using an acid. The pH adjusted effluent may then be air sparged for 30 minutes to drive off liberated CO₂. Following removal of CO₂, the pH of the sparged stream may be adjusted to about 9 to about 10 using NaOH.

Post-Oxidation Test Equipment

The advanced oxidation (AO) processes, consisting of oxidation with ozone (O₃), hydrogen peroxide (H₂O₂), ultraviolet light (UV), or combinations thereof, were performed in a bench scale reactor. The bench scale reactor consisted of a 0.5-1.0 liter glass cylinder containing an ozone diffuser tube and an ultraviolet (UV) light. The UV light source was either a low pressure mercury vapor bulb (15W) or a medium pressure mercury vapor bulb (150 W) complete with power supply and ballast. Ozone was generated using a one pound per day Welsbach ozone generator. Compressed air from a 1A cylinder was the source of dry gas for the ozone generator. The contents of the bench scale reactor were thoroughly mixed by the action of the applied ozone-containing gas and/or a magnetic stirrer. The AO process was conducted by filling the reactor with 0.3-0.7 liters of wet oxidation effluent. At the start of the test, H₂O₂ was added to the reactor for tests that involved H₂O₂. Tests involving O₃ started with the sparging of O₃ through the wastewater. If applicable, the mercury vapor bulb was switched on immediately after the first addition of the chemical oxidant. In the tests using O₃ gas, the dosage of O₃ was applied over the total duration of the test. At the conclusion of the test, the flow of O₃ gas was terminated, the UV bulb switched off, and catalase added to the effluent in which H₂O₂ was used. The catalase was added to destroy any residual H₂O₂. After completion of the test, the treated effluent was removed from the reactor.

### EXAMPLE XI

In order to evaluate post-oxidation treatment by advanced oxidation methods, a feed material containing 500 mg/L each of methane sulfonic acid (MSA) and methyl phosphonic acid (MPA) was prepared. The solution also contained 50 g/L trisodium phosphate, 11 g/L sodium fluoride, 0.1 g/L sodium formate and 50 g/L sodium carbonate. A sample of the feed material was treated with UV/O₃, as described above, and samples of the solution were analyzed for MSA and MPA at various time intervals during AO treatment.

TABLE XI
MSA And MPA Oxidation With Carbonate Using UV/O₃

| | | Test No. | | |
|---|---|---|---|---|
| | FEED | 37 | 38 | 39 |
| Time, minutes | — | 60 | 180 | 360 |
| O₃ Dosage, mg/L | — | 3,132 | 5,469 | 11,081 |
| MSA, mg/L | 433 | 403 | 191 | 98 |
| C—S DRE, % | — | 6.9 | 55.9 | 77.4 |
| MPA, mg/L | 484 | 300 | 9.8 | 2.7 |
| C—P DRE, % | — | 38 | 98 | 99.4 |

Addition of hydrogen peroxide at various intervals in the AO treatment did not improve destruction of MSA or MPA.

### EXAMPLE XII

The feed material of Example XI was prepared without sodium carbonate. A sample of the feed material was treated with UV/O₃, as described above, and samples of the solution were analyzed for MSA and MPA at various time intervals during AO treatment.

TABLE XII
MSA And MPA Oxidation Without Carbonate Using UV/O₃

| | | Test No. | | |
|---|---|---|---|---|
| | FEED | 40 | 41 | 42 |
| Time, minutes | — | 90 | 270 | 540 |
| O₃ Dosage, | — | 3,754 | 11,006 | 22,072 |
| mg/L | | | | |
| MSA, mg/L | 454 | 451 | 413 | 364 |
| C—S DRE, % | — | 6.7 | 9.0 | 19.8 |
| MPA, mg/L | 466 | 192 | 79.1 | <2.0 |
| C—P DRE, % | — | 58.8 | 83 | >99.6 |

MSA destruction was less effective with the carbonate removed from the solution, while MPA destruction was little affected by carbonate removal.

### EXAMPLE XIII

The advanced oxidation conditions chosen for treatment of the wet oxidation effluent from the four simulant solutions of examples IV-VII are as follows. The alkaline wet oxidation effluent was adjusted to pH 4-5 using concentrated hydrochloric acid. The sample was sparged with zero grade air for 30 minutes to remove any dissolved carbon dioxide. The pH of the sample was adjusted to between 9 and 10 using sodium hydroxide. The carbonate free wet oxidation effluent was placed in an AO vessel fitted with a medium pressure UV bulb. Oxygen gas containing 3% ozone was sparged through the effluent at 500 ml/min for 4.5 hours (270 minutes) with the UV bulb operating.

A summary of the results for wet oxidation of the four simulant solutions, followed by AO treatment with UV/O₃, is shown in Table XII.

TABLE XIII
Simulant Oxidations Performance Summary

| | Test No. | | | |
|---|---|---|---|---|
| | 43 | 44 | 45 | 46 |
| Feed Material | H in MEA | GB in MEA | DF | QL |
| Time, minutes | 60 | 180 | 360 | 180 |
| Temp., ° C. | 320 | 320 | 320 | 320 |
| Target | C—S Bond | C—P Bond | C—P Bond | C—P Bond |
| Compound | | | | |
| Overall | >99.97% | >97.8% | 99.88% | >97.52% |
| Oxidation DRE | | | | |

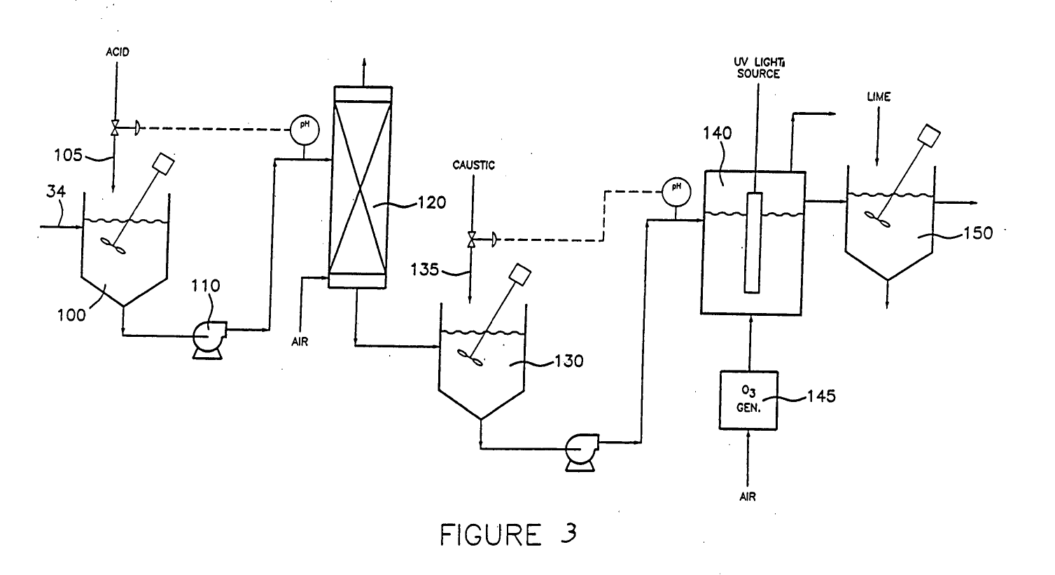

High C—S bond and C—P bond DRE was obtained using the two stage oxidation process. The flow scheme for a continuous flow wet oxidation system, shown in FIG. 2, is followed by treatment of the wet oxidation effluent by advanced oxidation employing UV/O₃, shown in FIG. 3. The wet oxidation system components are the same as described for FIG. 1.

In FIG. 3, the wet oxidation effluent flows to the advanced oxidation stage from the low pressure separator. Carbon dioxide is one of the products of oxidation in the wet air oxidation process. Due to the high pH of the wet oxidation effluent, the carbon dioxide formed remains in solution as a carbonate salt. Carbonate salts are known to interfere with the advanced oxidation treatment. Therefore, it is preferred that the carbonate salts be removed before the advanced oxidation treatment is carried out. The wet oxidation effluent from the wet air oxidation low pressure separator flows to an acid mix tank 100 where the pH is reduced to about 4 to 5 by addition of acid via an acid line 105 to liberate the carbon dioxide. Once the effluent pH is acidic, the liquor stream flows, via a pump 110, to the top of an air stripper column 120 where the liquid contacts air to strip the absorbed carbon dioxide gas from the liquid. Following the air stripper column 120, the liquid stream pH is raised to about 9 to 10 in another mix tank 130, by adding caustic via line 135, to facilitate the advanced oxidation treatment. The liquid stream then flows through a UV/ozone contact cell 140 where residual organic compounds are removed by oxidation. Ozone is produced using air as an oxygen source gas via an ozone generator 145.

Alternatively, the wet oxidation effluent from the wet air oxidation low pressure separator flows to a lime treatment stage where carbon dioxide is removed by precipitation as calcium carbonate. The resulting stream then enters the advanced oxidation treatment stage.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, if such features, systems, materials and/or methods are not mutually inconsistent, is included within the scope of the present invention.

In the claims (as well as in the specification above), all transitional phrases such as “comprising,” “including,” “carrying,” “having,” “containing,” “involving,” and the like are to be understood to be open-ended, i.e. to mean including but not limited to. Only the transitional phrases “consisting of” and “consisting essentially of” shall be closed or semi-closed transitional phrases, respectively, as set forth in the United States Patent Office Manual of Patent Examining Procedures, section 2111.03.

1. A wet oxidation process comprising:
providing an aqueous mixture including at least one compound having a carbon-hetero atom bond, the hetero atom selected from the group consisting of: phosphorus, sulfur, and arsenic;
maintaining the aqueous mixture pH between about 8 to about 14; and
oxidizing the aqueous mixture at an elevated temperature and superatmospheric pressure to substantially destroy the carbon-hetero atom bond of said at least one compound to form an alkaline oxidized mixture.

2. The process of claim 1, wherein the aqueous mixture comprises a second compound having a carbon-hetero atom bond, the hetero atom selected from the group consisting of phosphorus, sulfur, and arsenic.

3. The process of claim 1, wherein the pH of the aqueous mixture is maintained between about 9 and about 10.5

4. The process of claim 1, wherein the pH of the aqueous mixture is maintained at about 10 or higher.

5. The process of claim 1, wherein the aqueous mixture pH is maintained by adding a metal hydroxide.

6. The process of claim 5, wherein the metal hydroxide is an alkali metal hydroxide.

7. The process of claim 1, wherein the aqueous mixture is oxidized at a temperature of about 240° C. to about the critical temperature of water.

8. The process of claim 7, wherein the aqueous mixture is oxidized at a temperature of about 280° C. to about 350° C.

9. The process of claim 7, wherein the aqueous mixture is oxidized at a temperature of about 320° C.

10. The process of claim 7, wherein the aqueous mixture is oxidized at a pressure of at least about 33 atmospheres.

11. The process of claim 10, wherein the aqueous mixture is oxidized at a pressure of about 80 atmospheres to about 275 atmospheres.

12. The process of claim 10, wherein the aqueous mixture is oxidized for at least about 1 hour to about 8 hours.

13. The process of claim 11, wherein the aqueous mixture is oxidized for about 1 hour to about 6 hours.

14. The process of claim 12, wherein the aqueous mixture is oxidized for about 6 hours.

15. The process of claim 1, wherein the aqueous mixture is oxidized in a continuous process.

16. The process of claim 1, wherein the aqueous mixture is oxidized with an oxygen-containing gas.

17. The process of claim 16, wherein the oxygen-containing gas is selected from the group consisting of: air, oxygen-enriched air, and oxygen.

18. The process of claim 1, wherein oxidizing the aqueous mixture destroys at least about 98% of the carbon-hetero atom bonds of the at least one compound.

19. The of claim 18, wherein oxidizing the aqueous mixture destroys at least about 99% of the carbon-hetero atom bonds of said at least one compound.

20. The process of claim 1, wherein the aqueous mixture includes at least one halogen-containing compound.

21. The process of claim 1, further including adding at least one of a carbonate and a bicarbonate to the aqueous mixture.

22. The process of claim 21, wherein the carbonate is selected from the group consisting of sodium carbonate and potassium carbonate.

23. The process of claim 21, wherein the at least one of a carbonate and a bicarbonate is added to the aqueous mixture prior to oxidizing the aqueous mixture.

24. The process of claim 21, wherein the at least one of a carbonate and a bicarbonate is added after a portion of the aqueous mixture is oxidized.

25. The process of claim 1, further including adding oxidizable material to the aqueous mixture, wherein the oxidizable material has a carbonate as an oxidation product.

26. The process of claim 25, wherein the oxidizable material is a phenolic compound.

27. The process of claim 26, wherein the phenolic compound is selected from the group consisting of: phenol, cresol, and combinations thereof.

28. The process of claim 25, wherein the oxidizable material is a quinone.

29. The process of claim 28, wherein the quinone selected from the group consisting of: benzoquinone, hydroquinone, anthraquinone, and combinations thereof.

30. The process of claim 25, wherein the oxidizable material is added to the aqueous mixture prior to oxidizing the aqueous mixture.

31. The process of claim 25, wherein the oxidizable material is added after a portion of the aqueous mixture is oxidized.

32. A process for the destruction of carbon-hetero atom bonds comprising:
providing an aqueous mixture including at least one compound having a carbon-hetero atom bond, the hetero atom selected from the group consisting of: phosphorus, sulfur and arsenic;
maintaining the aqueous mixture pH between about 8 and about 14; and
oxidizing the aqueous mixture in a continuous process at a temperature of at least about 240° C. to less than about the critical temperature of water, and a pressure of at least about 33 atmospheres for a duration of about 1 hour to about 8 hours, to destroy at least about 95% of the carbon-hetero atom bonds of the at least one compound to form an alkaline oxidized mixture.

33. The process of claim 32, wherein the aqueous mixture is oxidized with an oxygen-containing gas.

34. The process of claim 33, wherein the oxygen-containing gas is selected from the group consisting of air, oxygen-enriched air, and oxygen.

35. The process of claim 32, wherein oxidizing the aqueous mixture destroys at least about 98% of the carbon-hetero atom bonds of the at least one compound.

36. The process of claim 35, wherein oxidizing the aqueous mixture destroys at least about 99% of the carbon-hetero atom bonds of the at least one compound.

37. The process of claim 32, wherein the aqueous mixture includes at least one halogen-containing compound.

38. The process of claim 32, wherein the pH of the aqueous mixture is maintained between about 9 to about 10.5.

39. The process of claim 32, wherein the aqueous mixture is oxidized at a temperature of between about 280° C. to about 350° C.

40. The process of claim 32, further comprising adding at least one of a carbonate and a bicarbonate to the aqueous mixture.

41. The process of claim 40, wherein the at least one of a carbonate and a bicarbonate is added to the aqueous mixture after a portion of the aqueous mixture is oxidized.

42. The process of claim 32, further comprising adding an oxidizable material to the aqueous mixture.

43. The process of claim 42, wherein the oxidizable material forms a carbonate when oxidized.

44. The process of claim 43, wherein the oxidizable material is a phenolic compound.

45. The process of claim 44, wherein the oxidizable material is phenol.

46. The process of claim 42, wherein the oxidizable material is added to the aqueous mixture after a portion of the aqueous mixture is oxidized.

47. A process for the destruction of carbon-hetero atom bonds comprising;
providing an aqueous mixture including at least one compound having a carbon-hetero atom bond, the hetero atom selected from the group phosphorus, sulfur and arsenic;
maintaining the aqueous mixture pH between about 8 and about 14;
oxidizing the aqueous mixture with a first oxidant to substantially destroy the carbon-hetero atom bond of said at least one compound to form a first alkaline oxidized mixture;
adjusting the first alkaline oxidized mixture pH to a range of about 3 to about 6 to produce an acidic first oxidized mixture; and
oxidizing the acidic first oxidized mixture with a second oxidant to destroy the carbon-hetero atom bond of any of the at least one compound remaining therein.

48. The process of claim 47, wherein the first oxidant is an oxygen-containing gas and the second oxidant is selected from the group hydrogen peroxide, ozone, and combinations thereof.

49. The process of claim 47, wherein the second oxidant is an iron-catalyzed hydrogen peroxide.

50. The process of claim 49, wherein the iron-catalyzed hydrogen peroxide is hydrogen peroxide catalyzed with a ferrous salt.

51. The process of claim 47, further comprising adjusting the pH of the acidic first oxidation mixture to between about 8 and about 10 prior to oxidation.

52. The process of claim 51, wherein the second oxidant is a combination of ozone and ultraviolet light.

53. The process of claim 47, wherein the aqueous mixture includes at least one halogen-containing compound.

54. The process of claim 47, wherein the aqueous mixture pH is adjusted with an alkali metal hydroxide.

55. The process of claim 47, wherein the aqueous mixture is oxidized at a temperature of at least about 240° C. to less than about the critical temperature of water, and a pressure of at least about 33 atmospheres, for at least about 1 hour to about 8 hours.

56. The process claim 47, wherein oxidation with the first and second oxidant destroys at least about 99% of the carbon-hetero atom bonds of said at least one compound.

57. The process of claim 47, further including adding an oxidizable material to the aqueous mixture prior to the oxidizing the aqueous material.

58. A system for treatment of compounds having carbon-hetero atom bonds, comprising:
a source of an aqueous mixture comprising at least one compound having a carbon-hetero atom bond;
a wet oxidation system fluidly connected to the source of the aqueous mixture; and
an alkali source fluidly connected to the source of aqueous mixture and upstream of the wet oxidation system.

59. The system of claim 58, wherein the carbon-hetero atom bond is selected from the group consisting of: phosphorus, sulfur, and arsenic.

60. The system of claim 58, further comprising a liquid effluent and a gas effluent of the wet oxidation system.

61. The system of claim 60, further comprising a second oxidation system fluidly connected to the liquid effluent.

62. The system of claim 61, further comprising an acidic source fluidly connected to the liquid effluent and upstream of the second oxidation system.

63. The system of claim 58, further comprising a source of at least one of a carbonate and a bicarbonate fluidly connected to the wet oxidation system.

64. The system of claim 58, further comprising a source of an oxidizable material fluidly connected to the wet oxidation system.

65. The system of claim 58 or 59 further comprising:
a separation unit fluidly connected to wet oxidation system and having a liquid effluent;
an acidic source fluidly connected to the liquid effluent to form an acidic influent;
a sparger fluidly connected to the acidic influent;
an alkali source fluidly connected to the acidic effluent and downstream of the sparger; and
a second oxidation system fluidly connected to the sparger downstream of the alkali source.

66. A wet air oxidation system, comprising;
a source of an aqueous mixture;
a wet oxidation system fluidly connected to the source of the aqueous mixture;
a source of an alkali fluidly connected to the source of the aqueous mixture and upstream of the wet air oxidation system; and
a source of carbonate fluidly connected to the wet air oxidation system downstream of the source of the aqueous mixture.

67. The wet air oxidation system of claim 66, wherein the source of carbonate is fluidly connected to the wet air oxidation system at an inlet for the aqueous mixture to the wet air oxidation system.

68. The wet air oxidation system of claim 66, wherein the source of carbonate is fluidly connected to the wet air oxidation system downstream of an inlet for the aqueous mixture to the wet air oxidation system.

69. The wet air oxidation system of claim 66, wherein the source of carbonate includes a chemical compound which forms a carbonate when oxidized.
